(19) United States
(12) Patent Application Publication
(10) Pub. No.: US 2007/0207513 A1
(43) Pub. Date: Sep. 6, 2007

(54) Methods, Products, and Kits for Identifying an Analyte in a Sample

(75) Inventors: Keld Sorensen, Austin, TX (US); Michaela Hoffmeyer, Cedar Park, TX (US)

Correspondence Address: DAFFER MCDANIEL LLP, P.O. BOX 684908, AUSTIN, TX, 78768, US

(73) Assignee: LUMINEX CORPORATION, Austin, TX (US)

(21) Appl. No.: 11/682,034

(22) Filed: Mar. 5, 2007

Related U.S. Application Data
(60) Provisional application No. 60/779,404, filed on Mar. 3, 2006.

(51) Int. Cl. G01N 33/53

(52) U.S. Cl. 435/7.92

(57) ABSTRACT

Methods, products, and kits for identifying an analyte in a sample are provided. One embodiment of a method for identifying an analyte in a sample includes combining the sample with a first reactant capable of specifically coupling to the analyte. The first reactant is coupled to beads. The method also includes combining additional reactant with the beads. The additional reactant is capable of specifically coupling to the analyte or a second reactant coupled to an analyte. An enzyme is attached to the additional reactant. In addition, the method includes combining a substrate with the beads. The substrate is capable of specifically interacting with the enzyme to form a modified substrate. If the substrate interacts with the enzyme attached to the beads via the additional reactant, the solubility of the substrate changes causing the modified substrate to bind to a surface of the beads and/or the reactants bound to the beads. The method further includes identifying the analyte in the sample by detecting the modified substrate bound to the surface of the beads and/or the reactants bound to the beads.

### PRIORITY CLAIM

The present application claims priority to U.S. Provisional Application No. 60/779,404 filed Mar. 3, 2006.

### BACKGROUND OF THE INVENTION

1. Field of the Invention

The present invention generally relates to methods, products, and kits for identifying an analyte in a sample. Certain embodiments relate to altering the solubility of a substrate via interaction of the substrate with an enzyme attached to a reactant to form a modified substrate such that if the reactant is coupled to an analyte (directly or indirectly) and if the analyte is bound to a bead, the altered solubility of the substrate causes the modified substrate to bind to a surface of the bead and/or reactants bound to the bead.

2. Description of the Related Art

The following description and examples are not admitted to be prior art by virtue of their inclusion in this section.

Optical systems have been and will increasingly be used to obtain measurements for a number of different samples such as biological samples. One particular optical-based system that is becoming increasingly important in the biological assay field is the flow cytometer, which allows scientists to examine a sample for a relatively large number of analytes in a relatively short amount of time. As with all optical systems, the sensitivity of the measurements of a flow cytometer is dependent in large part on the signal-to-noise ratio (SNR) that can be obtained using the flow cytometer. In particular, as the SNR increases, the measurement sensitivity increases.

The optical design and configuration of flow cytometers, like all optical systems, have, therefore, been developed taking into consideration the SNR requirements. For instance, one way that the SNR can be increased is to increase the brightness of the light source used to illuminate the particles being measured. In particular, as the amount of light available for measurements increases, the SNR of the measurements will generally increase. In this manner, high intensity light sources such as lasers are often used in flow cytometers. Other ways to increase the SNR of a flow cytometer may include selection and configuration of focusing optics, collecting optics (for collecting light scattered or emitted from the sample), and detectors included in the flow cytometer. Although improvements in the SNR of flow cytometers have been made since flow cytometers were first introduced, the search for the maximum SNR possible is an ongoing task.

Optimizing the materials that are measured in flow cytometers such as fluorescently dyed beads and fluorescently dyed reagents has also been explored as a means for increasing the SNR of flow cytometer measurements. However, the materials that are available for use in flow cytometer measurements are often somewhat limited. For instance, the materials must be compatible with the sample that will be examined. In other words, preferably, the materials of the beads and fluorescent dyes do not alter the sample being measured or vice versa. In addition, the materials are preferably compatible with the design of the flow cytometer. For instance, preferably, the fluorescent dyes are excited at the wavelength(s) of at least one light source of the flow cytometer. In addition, the material of the beads preferably is not altered by the wavelength(s) of light they will be exposed to by the flow cytometer. For at least these reasons, it may be more attractive and less complicated to try to increase the SNR of flow cytometer measurements by altering the optical design and configuration of the flow cytometer as opposed to altering the materials that will be used in the samples. Nevertheless, since altering the optical design and configuration of the flow cytometer can be expensive and complicated, increasing the SNR of flow cytometer measurements via the materials that are measured in flow cytometers continues to be explored.

Accordingly, it would be advantageous to develop methods, products, and kits for identifying an analyte in a sample by increasing the amount of a detectable material that can be coupled to a reaction product thereby increasing the detectable signal of the reaction product (i.e., by amplification of the signal) that can be measured by a flow cytometer or other measurement system.

### SUMMARY OF THE INVENTION

The following description of various method, product, and kit embodiments is not to be construed in any way as limiting the subject matter of the appended claims.

An embodiment relates to a method for identifying an analyte in a sample. The method includes combining the sample with a first reactant capable of specifically coupling to the analyte. The first reactant is coupled to beads. The method also includes combining an additional reactant with the beads. The additional reactant is capable of specifically coupling to the analyte or a second reactant coupled to the analyte. An enzyme is attached to the additional reactant. In addition, the method includes combining a substrate with the beads. The substrate is capable of specifically interacting with the enzyme to form a modified substrate. If the substrate interacts with the enzyme attached to the beads via the additional reactant, the solubility of the substrate changes causing the modified substrate to bind to a surface of the beads and/or the reactants bound to the beads. The method further includes identifying the analyte in the sample by detecting the modified substrate bound to the surface of the beads and/or the reactants bound to the beads. The method described above may include any other step(s) of any other method(s) described herein.

Another embodiment relates to a product of a method for identifying an analyte in a sample. The product includes a first reactant coupled to the analyte. The first reactant is also coupled to a bead. The product also includes an additional reactant coupled to the analyte or a second reactant coupled to the analyte. An enzyme is attached to the additional reactant. In addition, the product includes a modified substrate bound to a surface of the bead and/or the reactants bound to the bead due to an interaction between an initial substrate and the enzyme that produced a change in solubility of the initial substrate. The product may be further configured as described herein.

An additional embodiment relates to a kit configured for use in identifying an analyte in a sample. The kit includes a first reactant capable of specifically coupling to the analyte. The first reactant is coupled to beads. The kit also includes an additional reactant capable of specifically coupling to the analyte or a second reactant coupled to the analyte. An enzyme is attached to the additional reactant. In addition, the kit includes a substrate capable of specifically interacting with the enzyme to form a modified substrate. If the substrate interacts with the enzyme attached to the beads via the additional reactant, the solubility of the substrate changes causing the modified substrate to bind to a surface of the beads and/or to the reactants bound to the beads. The kit may be further configured as described herein.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a schematic diagram illustrating a side view of one embodiment of a method for identifying an analyte in a sample;

FIGS. 2-3 are schematic diagrams illustrating a cross-sectional view of various embodiments of a system configured to measure fluorescence of particles;

FIG. 4 includes representative confocal micrographs showing ELF® 97 alcohol, which is produced by the activity of calf intestinal alkaline phosphatase (CIAP) coupled to microspheres, bound to the surface of the microspheres and/or bound to reactants bound to the microspheres;

FIG. 5 includes representative confocal micrographs showing bovine serum albumin (BSA)-coupled microspheres in the presence of ELF® 97 alcohol; and

FIG. 6 includes representative epifluorescence micrographs showing that fluorescence intensity is proportional to the ELF® 97 alcohol crystal matrix complexity.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

The following description generally relates to methods, products, and kits for identifying an analyte in a sample in which the amount of a detectable material that can be coupled to a reaction product is increased thereby increasing the detectable signal of the reaction product that can be measured by a flow cytometer or other measurement system. In this manner, the methods, products, and kits described herein can be used for signal amplification in flow cytometer applications and any other applications described herein.

As used herein, the term “beads” is generally defined to include microspheres, particles, polystyrene beads, microparticles, gold nanoparticles, quantum dots, nanodots, nanoparticles, nanoshells, beads, microbeads, latex particles, latex beads, fluorescent beads, fluorescent particles, colored particles, colored beads, tissue, cells, micro-organisms, organic matter, or any non-organic matter. The beads may serve as vehicles for molecular reactions. Examples of appropriate beads are illustrated in U.S. Pat. No. 5,736,330 to Fulton, U.S. Pat. No. 5,981,180 to Chandler et al., U.S. Pat. No. 6,057,107 to Fulton, U.S. Pat. No. 6,268,222 to Chandler et al., U.S. Pat. No. 6,449,562 to Chandler et al., U.S. Pat. No. 6,514,295 to Chandler et al., U.S. Pat. No. 6,524,793 to Chandler et al., and U.S. Pat. No. 6,528,165 to Chandler, which are incorporated by reference as if fully set forth herein. The methods, products, and kits described herein may be used with or include any of the beads described in these patents. In addition, beads for use in flow cytometry may be obtained from manufacturers such as Luminex Corporation, Austin, Tex. The terms “particles,” “beads,” and “microspheres” are used interchangeably herein.

Turning now to the drawings, it is noted that the figures are not drawn to scale. In particular, the scale of some of the elements of the figures is greatly exaggerated to emphasize characteristics of the elements. It is also noted that the figures are not drawn to the same scale. Elements shown in more than one figure that may be similarly configured have been indicated using the same reference numerals.

One embodiment relates to a method for identifying an analyte in a sample. Although the method is described further herein with respect to identifying an analyte in a sample, the method can be used to identify one or more analytes in one or more samples in a single experiment (e.g., a multiplexed experiment). In addition, the method can be used to determine an amount of one or more analytes present in one or more samples in a single experiment. Additional examples of experiments and assays in which the method embodiments described herein may be used are illustrated in U.S. Pat. No. 5,981,180 to Chandler et al., U.S. Pat. No. 6,046,807 to Chandler, U.S. Pat. No. 6,139,800 to Chandler, U.S. Pat. No. 6,366,354 to Chandler, U.S. Pat. No. 6,411,904 to Chandler, U.S. Pat. No. 6,449,562 to Chandler et al., and U.S. Pat. No. 6,524,793 to Chandler et al., which are incorporated by reference as if fully set forth herein. The methods described herein may include any step(s) of any of the method(s) described in these patents.

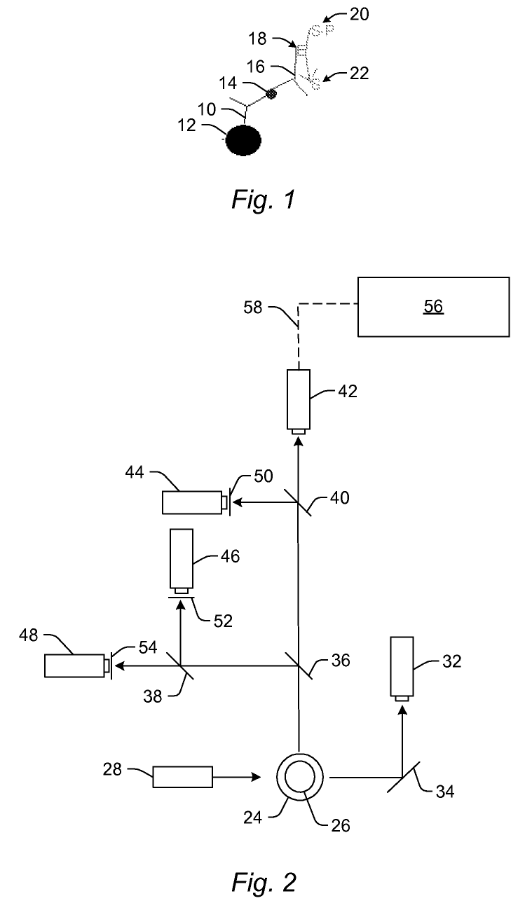

The method includes combining the sample with a first reactant capable of specifically coupling to the analyte. The first reactant is capable of specifically coupling to the analyte in that if more than one analyte is present in a sample, when the first reactant is combined with the sample, the first reactant will couple nearly exclusively with only one of the analytes. In this manner, the first reactant may be selected based on the analyte of interest in the sample (i.e., the analyte that is to be identified in the method). In one embodiment shown in FIG. 1, first reactant 10 is capable of specifically coupling to analyte 14. In other words, the first reactant is capable of capturing the analyte in a sample. In one such embodiment, first reactant 10 is an antibody. However, first reactant 10 may include any other appropriate reactant known in the art such as an antigen or an oligonucleotide. In one embodiment, if first reactant 10 is an antibody, analyte 14 may be an antigen. However, analyte 14 may include any other appropriate analyte known in the art such as an antibody and an oligonucleotide.

The first reactant is coupled to beads. In the embodiment shown in FIG. 1, first reactant 10 is coupled to bead 12. Bead 12 may have any suitable configuration known in the art. Combining the first reactant with the sample may include, for example, adding the beads having the first reactant attached thereto to the sample. However, the first reactant may be combined with the sample in any appropriate manner known in the art.

The first reactant may be coupled to beads that belong to one subset of a population of beads. Other reactants may be coupled to beads that belong to other subsets of the population. In this manner, each subset of beads may be coupled to a different first reactant, each of which specifically couples to a different analyte. As such, each subset of beads may be specifically designed for use in identifying only one analyte, and multiple analytes can be identified in a sample in a single experiment by combining the sample with more than one subset of beads within a population.

Optionally, the method includes combining a second reactant (not shown) with the beads. For example, the second reactant may be added to a mixture that includes the beads, the first reactant, and the sample. However, the second reactant may be combined with the beads in any appropriate manner known in the art. The second reactant is capable of specifically coupling to the analyte. The second reactant is capable of specifically coupling to the analyte in that if more than one analyte is present in a sample, when the second reactant is combined with the sample, the second reactant will couple nearly exclusively with only one of the analytes. In other words, the second reactant is capable of capturing the analyte in a sample. In this manner, the second reactant may be selected based on the analyte of interest in the sample (i.e., the analyte that is to be identified in the method). In one such embodiment, the second reactant is biotin. However, the second reactant may include any other appropriate reactant known in the art.

The method also includes combining an additional reactant with the beads. The additional reactant is capable of specifically coupling to the analyte or the second reactant coupled to the analyte. The additional reactant may be combined with the beads in any manner known in the art. For example, the additional reactant may be added to a mixture that includes the beads, the first reactant, optionally the second reactant, and the sample. The additional reactant is capable of specifically coupling to the analyte or the second reactant coupled to the analyte in that if more than one analyte or more than one second reactant is present in a sample, when the additional reactant is combined with the sample, the additional reactant will couple nearly exclusively with only one of the analytes or only one of the second reactants. The additional reactant may not be coupled to beads.

In the embodiment shown in FIG. 1, additional reactant 16 couples specifically to analyte 14. In one such embodiment, additional reactant 16 is an antibody. For example, the additional reactant may be a detection antibody. However, additional reactant 16 may include any other appropriate reactant known in the art such as an antigen or an oligonucleotide. The additional reactant may be selected based on the analyte of interest in the sample (i.e., the analyte that is to be identified in the method). In this manner, the assays described herein may involve forming a “traditional sandwich” for an immunoassay (e.g., a “sandwich” formed by first reactant 10, analyte 14, and additional reactant 16).

In another embodiment, if the second reactant is coupled to the analyte, the second reactant may be biotin, and the additional reactant may be streptavidin. In this manner, the “traditional sandwich” may be formed by first reactant 10, analyte 14, the second reactant, and additional reactant 16.

An enzyme is attached to the additional reactant. For instance, in the embodiment shown in FIG. 1, enzyme 18 is attached to additional reactant 16. An “enzyme” is defined herein to include any catalytic moiety known in the art and any molecule that can catalytically convert a substrate. Enzyme 18 may include, for example, horseradish peroxidase (HRP), alkaline phosphatase (Alk Phos), a traditional enzyme (e.g., a protein), a relatively low molecular weight molecule, a ribosome, RNA, or any other suitable molecule known in the art. The enzyme may be attached to the additional reactant directly or indirectly using any appropriate method known in the art. Different additional reactants (not shown) that are capable of specifically coupling to different analytes or different second reactants may be attached to different enzymes or the same enzyme.

The method further includes combining a substrate with the beads. The substrate is capable of specifically interacting with the enzyme to form a modified substrate. The substrate may be combined with the beads in any suitable manner known in the art. For instance, the substrate may be added to a mixture that includes the beads, the first reactant, the sample, optionally the second reactant, and the additional reactant. The substrate is capable of specifically interacting with the enzyme in that if more than one enzyme is present in a mixture (e.g., during a multi-analyte assay in which different enzymes are coupled to different additional reactants), when the substrate is combined with the mixture, the substrate will couple nearly exclusively with only one of the enzymes.

If the substrate interacts with the enzyme attached to the beads via the additional reactant, the solubility of the substrate changes causing the modified substrate to bind to a surface of the beads and/or the reactants bound to the beads. For example, an interaction between the substrate and the enzyme may render the initially soluble substrate insoluble or much less soluble in the solution in which the beads are disposed (i.e., the solution to which the substrate is added). In other words, the substrate is rendered insoluble or much less soluble by the enzymatic activity. In the embodiment shown in FIG. 1, substrate 20 (S-P) interacts with enzyme 18 attached to bead 12 via additional reactant 16. Substrate 20 is a soluble substrate. Interaction between enzyme 18 and substrate 20 produces modified substrate 22 (S), which is the substrate after processing by the enzyme and which is insoluble or less soluble than substrate 20. In one embodiment, substrate 20 may be a commercially available substrate such as ELF® 97 phosphate, which is commercially available from Invitrogen Corporation, Carlsbad, Calif. ELF® 97 phosphate is a phosphorylated dye that is freely water soluble when in its native form. If enzyme 18 is phosphatase, upon interaction of ELF® 97 phosphate with enzyme 18, the charged phosphate group of the substrate is cleaved off of the substrate thereby rendering the resulting new molecule (e.g., modified substrate 22) much less soluble than the initial molecule. The substrate may also include any other suitable substrate known in the art. Although substrate 20 may include a commercially available substrate or another suitable substrate known in the art, such substrates have never been used as described herein.

When the solubility of the substrate in the solution decreases, the modified substrate will be essentially “forced out” of the solution. In this manner, the reduced solubility substrate will migrate to a solid in the solution. In particular, when the solubility of the substrate is changed by the enzyme attached to the bead via the additional reactant, the bead itself (and reactants bound thereto) will be in relatively close proximity to the substrate (particularly compared to other beads in the solution, which are in reality substantially far from each other while the substrate is substantially close to the bead attached to the enzyme with which the substrate interacts, and compared to the vessel in which the solution is disposed). In other words, when the substrate is rendered insoluble or much less soluble by enzymatic activity, the closest object that it can “settle out” on is the bead and/or the reactants bound to the bead. As such, when the solubility of the substrate is reduced, the modified substrate will migrate to the bead thereby facilitating binding of the modified substrate to the surface of the bead and/or the reactants bound to the bead. In this manner, the modified substrate may be considered to precipitate out of solution, which facilitates binding of the modified substrate to the bead and/or the reactants bound to the bead. However, actual precipitation of the modified substrate is not required in the methods described herein.

Preferably, substrate 20 does not emit fluorescent light upon illumination, and modified substrate 22 coupled to the surface of the bead and/or the reactants bound to the bead emits fluorescent light when illuminated. Alternatively, substrate 20 may emit fluorescent light at one wavelength or wavelength band while modified substrate 22 emits fluorescent light at another wavelength or wavelength band that is sufficiently different than the wavelength or wavelength band of light emitted by the substrate. In this manner, fluorescent light emitted by the substrate will not interfere with detection of fluorescent light emitted by the modified substrate. As such, in the embodiments described herein, instead of a dye attached to the final reagent (e.g., the additional reactant) used in the assay, an enzyme is attached to the final reagent. After the additional reactant has been allowed to react with an analyte in a sample or a second reactant coupled to the analyte, which has been contacted with the beads described herein, the beads are incubated in a substrate that is less soluble when processed by the enzyme. As described above, when the substrate interacts with the enzyme, the substrate will be rendered less soluble thereby facilitating coupling of the modified substrate to the beads.

The embodiments described herein have a number of advantages over other methods and systems for identifying an analyte in a sample. For instance, there is an inherent lack of sensitivity in traditional assay technology in that dyes are bound to the particles and then counted to identify the analytes in a sample. In contrast, the embodiments described herein are able to enhance the signal produced by that amount of dye by many orders of magnitude. In other words, the embodiments described herein are configured for signal amplification. The problem with other systems and methods is the inherent limitation in dyes (i.e., a finite, relatively large amount of dye must be bound to the beads, then quantified by illumination with a laser and detection with a photomultiplier tube (PMT) or other similarly configured optical system). To the end user of such systems and methods, the sensitivity issue is the important one. The embodiments described herein afford greatly enhanced sensitivity that is expected to be on the order of several orders of magnitude larger than that of currently used systems and methods.

FIG. 1 also illustrates one embodiment of a product of a method for identifying an analyte in a sample. In particular, the product includes first reactant 10 coupled to analyte 14. First reactant 10 is also coupled to bead 12. The product also includes additional reactant 16 coupled to analyte 14. Alternatively, the additional reactant may be coupled to a second reactant (not shown) coupled to the analyte. Enzyme 18 is attached to the additional reactant. In addition, although modified substrate 22 is not shown in FIG. 1 bound to a surface of bead 12 and/or the reactants bound to the bead, the product includes modified substrate 22 bound to a surface of bead 12 and/or the reactants bound to the bead due to interaction between an initial substrate (e.g., substrate 20) and enzyme 18 that produced a change in solubility of initial substrate 20. The product of the method may be further configured as described herein.

The method also includes identifying the analyte in the sample by detecting the modified substrate bound to the surface of the beads and/or the reactants bound to the beads. Identifying the analyte in the sample may be performed by detecting light emanating from the product of the method due to illumination of the product. In particular, illumination of the product may cause the modified substrate bound to the surface of the bead to emit fluorescent light. The fluorescent light may be detected (e.g., by one of the systems described herein) and used as the “reporter signal.” For example, the modified substrate may be known (e.g., a priori or via experimentation) to emit fluorescent light having one or more specific characteristics. Therefore, measurements of beads that exhibit emission of fluorescent light having the one or more specific characteristics may be used to identify the beads to which the modified substrate has been bound (via the surface of the beads and/or the reactants bound to the beads). The modified substrate bound to the beads (via the surface of the beads and/or the reactants bound to the beads) indicates that a reaction product (e.g., a first reactant-analyte—(optional second reactant)—additional reactant reaction product) has been formed on the beads. The beads themselves may also be configured to emit fluorescent light having one or more characteristics that are indicative of the first reactant coupled to the beads. The modified substrate and the beads may or may not be excited at the same wavelength or wavelength band. Measurements of the fluorescence emitted by the beads themselves can be used to determine an identity of the reaction that has taken place on the beads. The identity of the reaction can then be used to determine the identity of the analyte in the sample.

In another embodiment, each of the additional reactants that is capable of specifically coupling to an analyte in a sample or a second reactant coupled to the analyte is attached to a different enzyme, and each of the different substrates (combined with the beads in a mixture) is capable of specifically interacting with only one of the enzymes. The substrates capable of interacting with different enzymes may be selected such that upon interaction with the corresponding enzyme, the different modified substrates are capable of emitting fluorescent light having one or more different characteristics such as different wavelength(s), different wavelength band(s), different intensities, etc. The different modified substrates may or may not be excited at the same wavelength or wavelength band(s). In this manner, detecting the modified substrate bound to the beads (via the surface of the beads and/or the reactants bound to the beads) may include illuminating the beads and detecting fluorescence emitted from the beads. The detected fluorescence may be used to determine which substrates have been modified during the method. In addition, the identity of a substrate that has been modified can be used to determine an identity of a additional reactant that has reacted with an analyte or a second reactant coupled to an analyte, coupled to a bead via a first reactant, since nearly all of the substrate molecules that interact with an enzyme attached to a bead, indirectly via the first reactant-analyte—(optional second reactant)—additional reactant complex, will bind to that bead (via the surface of that bead or the reactants bound to that bead). In this manner, detecting a modified substrate on a bead indicates that a first reactant-analyte—(optional second reactant)—additional reactant complex has been formed thereby indicating that the analyte is present in the sample. In such embodiments, the beads may or may not be configured to exhibit different light scattering characteristics and/or different fluorescence emission characteristics since the identity of the analyte in the sample can be determined from the fluorescence emitted by the modified substrate.

The method embodiments described herein may also include determining an amount of the analyte in the sample. The amount of the analyte in the sample may be determined as described further herein.

An additional embodiment relates to a kit configured for use in identifying an analyte in a sample. The kit includes a first reactant capable of specifically coupling to the analyte. The first reactant may include any of the first reactants described herein. In addition, the first reactant may be further configured as described herein. The first reactant is coupled to beads. The beads may include any of the beads described herein. In addition, the beads may be further configured as described herein.

The kit also includes an additional reactant capable of specifically coupling to the analyte or a second reactant coupled to the analyte. The kit may include the second reactant, which may include any of the second reactants described herein. The additional reactant may include any of the additional reactants described herein. In addition, the additional reactant may be further configured as described herein. An enzyme is attached to the additional reactant. The enzyme may include any of the enzymes described herein. The enzyme may also be further configured as described herein.

The kit further includes a substrate capable of specifically interacting with the enzyme to form a modified substrate. The substrate may include any of the substrates described herein. If the substrate interacts with the enzyme attached to the beads via the additional reactant, the solubility of the substrate changes causing the modified substrate to bind to a surface of the beads and/or to the reactants bound to the beads. The substrate may be further configured as described herein. Each component of the kit, including the first reactant, the optional second reactant, the additional reactant, and the substrate, may be contained in a separate vessel. The kit may also include any other components that can be used to perform a method described herein.

FIG. 2 illustrates one embodiment of a system configured to measure fluorescence of particles. The embodiment of the system shown in FIG. 2 is configured as a flow cytometer. In FIG. 2, the system is shown along a plane through the cross-section of cuvette 24 through which particles 26 flow. In one example, the cuvette may be a standard fused-silica cuvette such as that used in standard flow cytometers. Any other suitable type of viewing or delivery chamber, however, may also be used to deliver the sample for analysis.

The system includes an illumination subsystem configured to illuminate particles 26 with light. The illumination subsystem includes light source 28. In one embodiment, light source 28 is a laser. The laser may be any suitable laser known in the art. The light source may be configured to emit light having one or more wavelengths such as blue light or green light. In other embodiments, the light source includes one or more non-laser light sources (not shown) selected from the group consisting of light emitting diodes (LEDs), arc lamps, fiber illuminators, and light bulbs. The non-laser light source(s) may include any suitable non-laser light source(s) known in the art. The illumination subsystem may also include more than one light source. In one embodiment, the light sources may be configured to illuminate the particles with light having different wavelengths or wavelength bands (e.g., blue light and green light). In some embodiments, the light sources may be configured to illuminate the particles at different directions. In addition, the illumination subsystem may include one or more lasers and/or one or more non-laser light sources. Light source 28 may include any other appropriate light source known in the art. In some embodiments, the system may include one or more lenses (not shown) configured to focus light from the light source onto the particles or the flowpath.

The illumination causes the particles or a material (e.g., a modified substrate) attached thereto or incorporated therein to emit fluorescent light having one or more wavelengths or wavelength bands. In some embodiments, particles 26 themselves are configured to emit fluorescence. All such fluorescence is generally referred to in further description provided herein as “fluorescence emitted by the particles.”

Light scattered forwardly from the particles may be directed to detection system 32 by folding mirror 34 or another suitable light directing component. Alternatively, detection system 32 may be placed directly in the path of the forwardly scattered light. In this manner, the folding mirror or other light directing components may not be included in the system. In one embodiment, the forwardly scattered light may be light scattered by the particles at an angle of about 180° from the direction of illumination by light source 28, as shown in FIG. 2. The angle of the forwardly scattered light may not be exactly 180° from the direction of illumination such that incident light may not impinge upon the photosensitive surface of the detection system. For example, the forwardly scattered light may be light scattered by the particles at angles less than or greater than 180° from the direction of illumination (e.g., light scattered at an angle of about 170°, about 175°, about 185°, or about 190°).

Light scattered by the particles at an angle of about 90° from the direction of illumination may also be collected. Light scattered by the particles can also or alternatively be collected at any angle or orientation. In one embodiment, this scattered light may be separated into more than one beam of light by one or more beamsplitters or dichroic mirrors. For example, light scattered at an angle of about 90° to the direction of illumination may be separated into two different beams of light by beamsplitter 36. The two different beams of light may be separated again by beamsplitters 38 and 40 to produce four different beams of light. Each of the beams of light may be directed to a different detection system, which may include one or more detectors. For example, one of the four beams of light may be directed to detection system 42. Detection system 42 may be configured to detect light scattered by the particles.

Scattered light detected by detection system 32 and/or detection system 42 may generally be proportional to the volume of the particles that are illuminated by the light source. Therefore, output signals of detection system 32 and/or output signals of detection system 42 may be used to determine a diameter of the particles that are in the illumination zone or detection window. In addition, the output signals of detection system 32 and/or detection system 42 may be used to identify more than one particle that are stuck together or that are passing through the illumination zone at approximately the same time. Therefore, such particles may be distinguished from other sample particles and calibration particles.

The system also includes a detection subsystem configured to generate output signals responsive to the fluorescence emitted by the particles. For example, the other three beams of light may be directed to detection systems 44, 46, and 48. Detection systems 44, 46, and 48 may be configured to detect fluorescence emitted by the particles. Each of the detection systems may be configured to detect fluorescence of a different wavelength or a different range of wavelengths. For example, one of the detection systems may be configured to detect green fluorescence. Another of the detection systems may be configured to detect yellow-orange fluorescence, and the other detection system may be configured to detect red fluorescence.

In some embodiments, spectral filters 50, 52, and 54 may be coupled to detection systems 44, 46, and 48, respectively. The spectral filters may be configured to block fluorescence of wavelengths other than that or those which the detection systems are configured to detect. In addition, one or more lenses (not shown) may be optically coupled to each of the detection systems. The lenses may be configured to focus the scattered light or emitted fluorescence onto a photosensitive surface of the detectors.

The detector's output current is proportional to the fluorescent light impinging on it and results in a current pulse. The current pulse may be converted to a voltage pulse, low pass filtered, and then digitized by an A/D converter (not shown). Processor 56 such as a digital signal processor (DSP) integrates the area under the pulse to provide a number which represents the magnitude of the fluorescence. As shown in FIG. 2, processor 56 may be coupled to detector 42 via transmission medium 58. Processor 56 may also be coupled to detector 42 indirectly via transmission medium 58 and one or more other components (not shown) such as the A/D converter. The processor may be coupled to other detectors of the system in a similar manner. Processor 56 may be further configured as described herein.

In some embodiments, the output signals generated from fluorescence emitted by the particles may be used to determine an identity of the particles and information about a reaction taken or taking place on the surface of the particles. For example, output signals of two of the detection systems may be used to determine an identity of the particles, and output signals of the other detection system may be used to determine a reaction taken or taking place on the surface of the particles. Therefore, the selection of the detectors and the spectral filters may vary depending on the type of dyes or fluorophores incorporated into or bound to the particles and/or the reaction being measured (i.e., the enzymes attached to the reactants involved in the reaction and the modified substrates produced by interaction between the enzymes and the substrate).

The detection systems that are used to determine an identity of the sample particles (e.g., detection systems 44 and 46) may be avalanche photodiode (APDs), PMTs, or another type of photodetector. The detection system that is used to identify a reaction taken or taking place on the surface of the particles (e.g., detection system 48) may be a PMT, an APD, or another type of photodetector. The measurement system may be further configured as described herein.

Although the system of FIG. 2 is shown to include two detection systems having two different detection windows for distinguishing between particles having different dye characteristics, it is to be understood that the system may include more than two such detection windows (i.e., 3 detection windows, 4 detection windows, etc.). In such embodiments, the system may include additional beamsplitters and additional detection systems having other detection windows. In addition, spectral filters and/or lenses may be coupled to each of the additional detection systems.

In another embodiment, the system may include two or more detection systems configured to distinguish between modified substrates that are coupled to the particles (via the surface of the particles and/or the reactants bound thereto). The different modified substrates may have fluorescence emission characteristics that are different than the fluorescence emission characteristics of the particles.

Additional examples of measurement systems that may be used to identify an analyte as described herein are illustrated in U.S. Pat. No. 5,981,180 to Chandler et al., U.S. Pat. No. 6,046,807 to Chandler, U.S. Pat. No. 6,139,800 to Chandler, U.S. Pat. No. 6,366,354 to Chandler, U.S. Pat. No. 6,411,904 to Chandler, U.S. Pat. No. 6,449,562 to Chandler et al., and U.S. Pat. No. 6,524,793 to Chandler et al., which are incorporated by reference as if fully set forth herein. The measurement system described herein may also be further configured as described in these patents. The system shown in FIG. 2 may be further configured as described herein with respect to other systems and embodiments. In addition, the system shown in FIG. 2 has all of the advantages of other embodiments described herein.

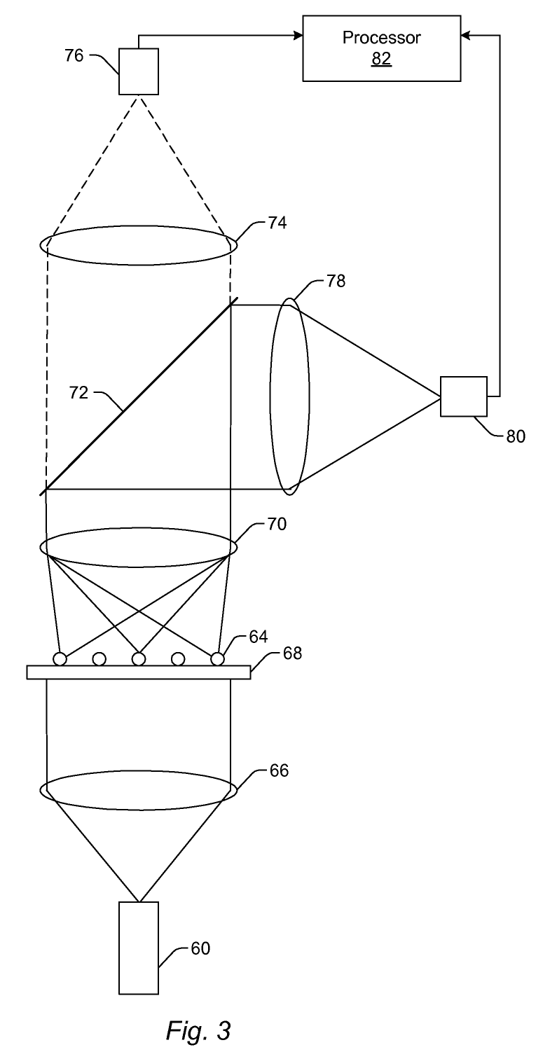

Another embodiment of a system configured to measure fluorescence of particles is shown in FIG. 3. The system shown in FIG. 3 may be used in applications such as multi-analyte measurement of a sample. This embodiment of the system is configured as a fluorescence imaging system. The system includes an illumination subsystem configured to illuminate the particles with light.

In one embodiment, the illumination subsystem includes light source 60. Light source 60 may include one or more light sources such as any suitable LEDs, lasers, arc lamps, fiber illuminators, light bulbs, incandescent lamps, or any other suitable light sources known in the art. In addition, the illumination subsystem may include more than one light source (not shown), each of which is configured to generate light of at least one wavelength or at least one wavelength band. One example of an appropriate combination of light sources for use in the system shown in FIG. 3 includes, but is not limited to, two or more LEDs.

Light generated by more than one light source may be combined into a common illumination path by a beamsplitter (not shown) or any other suitable optical element known in the art such that light from the light sources may be directed to the particles simultaneously. Alternatively, the imaging subsystem may include an optical element (not shown) such as a reflecting mirror and a device (not shown) configured to move the optical element into and out of the illumination path depending on which light source is used to illuminate the particles. In this manner, the light sources may be used to sequentially illuminate the particles with different wavelengths or wavelength bands of light. The light source(s) may also illuminate the substrate from above (not shown), rather than from below the substrate.

The light source(s) may be selected to provide light at wavelength(s) or wavelength band(s) that will cause the particles or materials coupled thereto (e.g., a modified substrate) or incorporated therein to emit fluorescence. For instance, the wavelength(s) or wavelength band(s) may be selected to excite fluorophores, fluorescent dyes, or other fluorescent materials incorporated into the particles and/or coupled to a surface of the particles. In this manner, the wavelength(s) or wavelength band(s) may be selected such that the particles emit fluorescence that is used for classification of the particles. In addition, the wavelength(s) or wavelength band(s) may be selected to excite fluorophores, fluorescent dyes, other fluorescent materials, or the modified substrates described herein coupled to the surface of the particles and/or coupled to the particles via a reagent on the surface of the particles. As such, the wavelength(s) or wavelength band(s) may be selected such that the particles emit fluorescence that is used to detect and/or quantify reaction(s) that have taken place on the surface of the particles.

As shown in FIG. 3, the illumination subsystem may include optical element 66 that is configured to direct light from the light source to plate 68 on which particles 64 are immobilized. In one example, optical element 66 may be a collimating lens. However, optical element 66 may include any other appropriate optical element that can be used to image light onto plate 68. In addition, although the optical element is shown in FIG. 3 as a single optical element, it is to be understood that optical element 66 may include more than one refractive element. Furthermore, although optical element 66 is shown in FIG. 3 as a refractive optical element, it is to be understood that one or more reflective and/or diffractive optical elements may be used (possibly in combination with one or more refractive optical elements) to image light onto plate 68. In addition, although optical element 66 is shown in FIG. 3 to image light onto plate 68 at a substantially normal angle of incidence, it is to be understood that the system may be configured to direct light to plate 68 at an oblique angle of incidence.

Particles 64 may include any of the particles described above. Plate 68 may include any appropriate plate known in the art. The particles immobilized on plate 68 may be disposed in an imaging chamber (not shown) or any other device for maintaining a position of plate 68 and particles 64 immobilized thereon with respect to the illumination subsystem. The device for maintaining a position of plate 68 may also be configured to alter a position of the plate (e.g., to focus the illumination onto the plate) prior to imaging.

Immobilization of the particles on the plate may be performed using magnetic attraction, a vacuum filter plate, or any other appropriate method known in the art. Examples of methods and systems for positioning microspheres for imaging are illustrated in U.S. patent application Ser. No. 11/270,786 to Pempsell filed Nov. 9, 2005, which is incorporated by reference as if fully set forth herein. The particle immobilization method itself is not particularly important to the method and systems described herein. However, the particles are preferably immobilized such that the particles do no move perceptibly during the detector integration period, which may be multiple seconds long.

The system shown in FIG. 3 also includes a detection subsystem that is configured to generate output signals responsive to the fluorescence emitted by the particles. For example, as shown in FIG. 3, the detection subsystem may include optical element 70 and dichroic beamsplitter 72. Optical element 70 is configured to collect and collimate light from plate 68 and particles 64 immobilized thereon and to direct the light to beamsplitter 72. Optical element 70 may be further configured as described above with respect to optical element 66. Beamsplitter 72 may include any appropriate beamsplitter known in the art. Beamsplitter 72 may be configured to direct light from optical element 70 to different detectors based on the wavelength of the light. For example, light having a first wavelength or wavelength band may be transmitted by beamsplitter 72, and light having a second wavelength or wavelength band different than the first may be reflected by beamsplitter 72.

The detection subsystem may also include optical element 74 and detector 76. Light transmitted by beamsplitter 72 may be directed to optical element 74. Optical element 74 is configured to focus the light transmitted by the beamsplitter onto detector 76. The detection subsystem may further include optical element 78 and detector 80. Light reflected by beamsplitter 72 may be directed to optical element 78. Optical element 78 is configured to focus the light reflected by the beamsplitter onto detector 80. Optical elements 74 and 78 may be configured as described above with respect to optical element 66.

Detectors 76 and 80 may include, for example, charge coupled device (CCD) detectors or any other suitable imaging detectors known in the art such as CMOS detectors, two-dimensional arrays of photosensitive elements, time delay integration (TDI) detectors, etc. In some embodiments, a detector such as a two-dimensional CCD imaging array may be used to acquire an image of substantially an entire plate or of all particles immobilized on a plate simultaneously. The number of detectors included in the system may be equal to the number of wavelengths or wavelength bands of interest such that each detector is used to generate images at one of the wavelengths or wavelength bands.

Each of the images generated by the detectors may be spectrally filtered using an optical bandpass element (not shown) or any other suitable optical element known in the art, which is disposed in the light path from the beamsplitter to the detectors. A different filter “band” may be used for each captured image. The detection wavelength center and width for each wavelength or wavelength band at which an image is acquired may be matched to the fluorescent emission of interest, whether it is used for particle classification or the reporter signal.

In this manner, the detection subsystem of the system shown in FIG. 3 is configured to generate multiple images at different wavelengths or wavelength bands simultaneously. Although the system shown in FIG. 3 includes two detectors, it is to be understood that the system may include more than two detectors (e.g., three detectors, four detectors, etc.). As described above, each of the detectors may be configured to generate images at different wavelengths or wavelength bands simultaneously by including one or more optical elements for directing light at different wavelengths or wavelength bands to the different detectors simultaneously. In addition, although the system is shown in FIG. 3 to include multiple detectors, it is to be understood that the system may include a single detector. The single detector may be used to generate multiple images at multiple wavelengths or wavelength bands sequentially. For example, light of different wavelengths or wavelength bands may be directed to the substrate sequentially, and different images may be generated during illumination of the substrate with each of the different wavelengths or wavelength bands. In another example, different filters for selecting the wavelength or wavelength band of light directed to the single detector may be altered (e.g., by moving the different filters into and out of the imaging path) to generate images at different wavelengths or wavelength bands sequentially.

The detection subsystem shown in FIG. 3, therefore, is configured to generate a plurality or series of images representing the fluorescent emission of particles 64 at several wavelengths of interest. In addition, the system may be configured to supply a plurality or series of digital images representing the fluorescence emission of the particles to a processor (i.e., a processing engine). In one such example, the system may include processor 82. Processor 82 may be configured to acquire (e.g., receive) image data from detectors 76 and 80. For example, processor 82 may be coupled to detectors 76 and 80 in any suitable manner known in the art (e.g., via transmission media (not shown), each coupling one of the detectors to the processor, via one or more electronic components (not shown) such as analog-to-digital converters, each coupled between one of the detectors and the processor, etc.).

Preferably, processor 82 is configured to process and analyze the images to determine one or more characteristics of particles 64 such as a classification of the particles and information about a reaction taken place on the surface of the particles. The processor may be configured to determine the classification and this information as described further herein. The one or more characteristics may be output by the processor in any suitable format such as a data array with an entry for fluorescent magnitude for each particle for each wavelength or wavelength band.

Processor 82 may be a processor such as those commonly included in a typical personal computer, mainframe computer system, workstation, etc. In general, the term “computer system” may be broadly defined to encompass any device having one or more processors, which executes instructions from a memory medium. The processor may be implemented using any other appropriate functional hardware. For example, the processor may include a DSP with a fixed program in firmware, a field programmable gate array (FPGA), or other programmable logic device (PLD) employing sequential logic “written” in a high level programming language such as very high speed integrated circuits (VHSIC) hardware description language (VHDL). In another example, program instructions (not shown) executable on processor 82 may be coded in a high level language such as C#, with sections in C++ as appropriate, ActiveX controls, JavaBeans, Microsoft Foundation Classes (“MFC”), or other technologies or methodologies, as desired. The program instructions may be implemented in any of various ways, including procedure-based techniques, component-based techniques, and/or object-oriented techniques, among others.

The system shown in FIG. 3 may be further configured as described herein with respect to other systems and embodiments. In addition, the system shown in FIG. 3 has all of the advantages of other embodiments described herein.

The following examples are included herein to illustrate that the embodiments described herein allow a vastly increased fluorescent signal to be generated (e.g., on the bead surface) thereby illustrating a reduction to practice. In particular, the following examples illustrate signal amplification for Luminex beads using enzymatically generated fluorescent materials. It is noted that due to the nature of the excitation wavelength in the current production version of the Luminex instrument, the signal of ELF® 97 cannot be detected by this instrument. However, the excitation wavelength can be customized for future instruments, and other substrates can be generated with wavelengths that more closely match currently used instruments. Nevertheless, the results presented herein clearly illustrate that the enzymatic deposition of materials capable of emitting fluorescent light will have a clearly higher signal than the current methodology of binding a single fluor molecule per binding event. The following examples are not to be construed as limiting embodiments of the invention and are included herein for example purposes only.

### EXAMPLE 1

### CIAP Coupling to Carboxylated Microspheres

Calf intestinal alkaline phosphatase (CIAP, Invitrogen), supplied in a Tris-based buffer, was dialyzed into 100 mM 2-(N-morpholino)ethanesulfonic acid (MES) pH 6.0 using Zeba™ Desalt Columns, which were obtained from Pierce Biotechnology, Inc., Rockford, Ill., before microsphere coupling. Standard, two-step carbodiimide reaction chemistry was used to couple CIAP to microspheres. Briefly, 5×10⁶ of the stock carboxylated microspheres (obtained from Luminex) were washed in water and resuspended in 100 mM monobasic sodium phosphate pH 6.2. The microspheres were activated by addition of 50 mg/mL N-hydroxysulfosuccinimide (Sulfo-NHS, obtained from Pierce) followed by 50 mg/mL 1-Ethyl-3-[3-dimethylaminopropyl]carbodiimide hydrochloride (EDC, obtained from Pierce) and allowed to incubate at room temperature for 20 minutes protected from light. Following incubation, the activated microspheres were washed twice and resuspended in 100 mM MES pH 6.0. 25 μg of CIAP was added to the activated microspheres, and the reaction volume brought to 0.5 mL with 100 mM MES pH 6.0. The reaction was protected from light and allowed to incubate for 2 hours with mixing at room temperature. The CIAP-coupled microspheres were then washed three times and resuspended in 100 mM MES pH 6.0. CIAP-coupled microspheres were enumerated and stored protected from light at 4° C. Coupling was confirmed by detection with biotinylated anti-CIAP rabbit polyclonal antibody (AbCAM) over a range of 0.0625 μg/mL to 4 μg/mL.

### EXAMPLE 2

### ELF® 97 Phosphate Reaction Conditions

A variety of ELF® 97 phosphate (obtained from Invitrogen) reactions were performed to determine the optimal conditions for microsphere coupled-CIAP generated ELF® 97 alcohol interaction with the microsphere surface and/or reactants bound thereto. All reactions were performed in 1× Tris-ethylenediaminetetraacetic acid (Tris-EDTA or TE) at 37° C. for 1 hour with agitation (1150 rpm) in a total reaction volume of 100 μL. Two different concentrations of microsphere-coupled CIAP were tested. Reactions were performed using either 200,000 or 5,000 CIAP-coupled microspheres. Three concentrations of ELF® 97 phosphate were also tested at each CIAP-coupled microsphere concentration: 500 μM, 250 μM, and 125 μM. ELF® 97 phosphate was filtered using 0.2 μm ELF® spin filters (obtained from Invitrogen) prior to use in every reaction to remove any preformed ELF® 97 alcohol crystals, as per the manufacturer's recommendation. ELF® 97 alcohol (obtained from Invitrogen) without CIAP-coupled microspheres was used as a positive control at a concentration of 100 μM. Bovine serum albumin (BSA)-coupled microspheres (200,000) were mixed with ELF® 97 alcohol at a final concentration of 100 μM to investigate the specificity of ELF® 97 alcohol interaction with the bead surface. Negative controls containing no CIAP-coupled microspheres but with ELF® 97 phosphate were also performed. 8 μL of each reaction was spotted onto a standard microscope slide. About 30 μL of Fluoromount-G (obtained from SouthernBiotech, Birmingham, Ala.) mounting agent was placed over the reaction spot and a #1 coverslip added. The slides were dried overnight and sealed thoroughly with nail polish prior to imaging.

### EXAMPLE 3

### Confocal Imaging

The Leica SP2 ABOS confocal microscope located in the Institute for Cellular and Molecular Biology Core Facility at the University of Texas at Austin was employed to examine the interaction of fluorescent ELF® 97 alcohol with the surface of the CIAP-coupled microspheres. ELF® 97 alcohol was excited in the ultraviolet (UV) region (about 350 nm), and emission was detected between 500 nm and 550 nm in the yellow-green region. The internal dyes of the carboxylated microspheres were excited at 635 nm, and emission was detected from 660 nm and 710 nm. For most reactions, 2 40× objective fields were imaged. Through-focus series were generated for some samples to examine the 3-dimensional structure of the ELF® 97 alcohol crystal matrix on the microspheres. The confocal settings varied with the sample imaged since the intensity of the ELF® 97 alcohol signal was proportional to the size of the ELF® 97 alcohol crystal formed at the microsphere surface. Furthermore, signal quantification was not a priority.

### EXAMPLE 4

### Epifluorescence Imaging

The Leica DM IRBE inverted fluorescence microscope located in the Institute for Cellular and Molecular Biology Core Facility at the University of Texas at Austin was utilized to examine the interface between the fluorescent ELF® 97 alcohol product and the CIAP-coupled microsphere surface. ELF® 97 was imaged using the 4′,6-diamidino-2-phenylindole (DAPI) fluorescence filter cube, while the internal dyes of the microspheres were imaged using the Cy5 fluorescence filter cube. For most reactions, 3 40× fields were captured with the Leica DFC350FX camera. Exposure time and gain settings again varied for each reaction since the ELF® 97 alcohol signal was proportional to the size of the ELF® 97 alcohol crystal formed at the microsphere surface. For experiments designed to compare and quantify fluorescence intensity between reactions containing differing concentrations of ELF® 97 phosphate, camera settings were held constant.

### EXAMPLE 5

### Generation of ELF® 97 Alcohol by CIAP-Coupled Microspheres

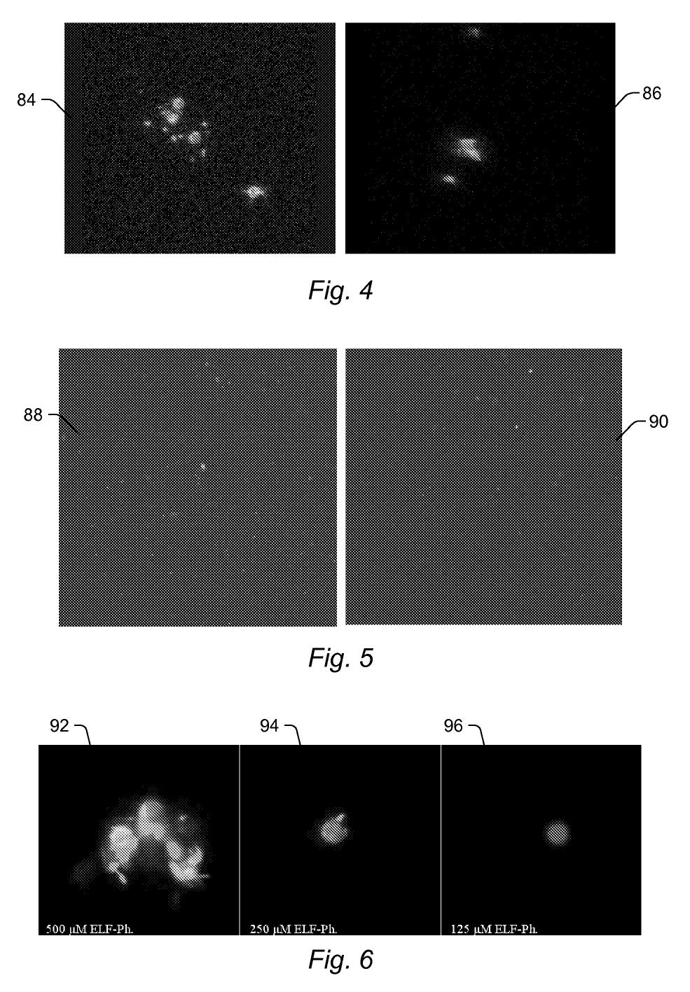

ELF® 97 phosphate experiments were performed and viewed under the fluorescence confocal microscope to ascertain if the ELF® 97 alcohol produced by the action of CIAP coupled to the microsphere would bind to the microsphere surface and/or reactants bound thereto. FIG. 4 shows representative free max projections of the through-focus series of two reactions, differing only in the number of CIAP-coupled microspheres.

Images 84 and 86 shown in FIG. 4 are representative confocal micrographs of free max projections of through-focus series at 400×. Reaction conditions for image 84 shown in FIG. 4 were 200,000 CIAP-microspheres, 500 μM ELF® 97 Phosphate, 1×TE, 37° C. 1 hr, 1150 rpm, light-protected, 100 μL reaction volume. Reaction conditions for image 86 shown in FIG. 4 were 5,000 CIAP-microspheres, 500 μM ELF® 97 Phosphate, 1×TE, 37° C. 1 hr, 1150 rpm, light-protected, 100 μL reaction volume.

ELF® 97 alcohol (green) is produced by the enzymatic activity of the CIAP-coupled microspheres (red) and does interact with the microspheres' surface and/or reactants bound thereto. Furthermore, the ELF® 97 alcohol crystal matrix is 3-dimensional and encompasses the surface area of the CIAP-coupled microspheres, as seen by confocal through-focus series.

However, it appears that the ELF® 97 alcohol crystal structure induced some aggregation of the beads. Reducing the bead concentration to that used in a typical reaction (5,000 beads per 100 μL) reduced aggregation without effecting ELF® 97 alcohol interaction with the bead surface (image 84 versus image 86) and/or reactants bounds thereto. Aggregation is therefore likely due to the interaction of bound ELF® 97 alcohol crystals between neighboring microspheres. Additionally, decreasing the ELF® 97 phosphate substrate concentration resulted in less ELF® 97 alcohol bound to the CIAP-coupled microsphere surface and/or reactants bound thereto.

### EXAMPLE 6

### Specificity of ELF® 97 Alcohol Interaction with Protein Coupled Microspheres

To determine if the interaction of the ELF® 97 alcohol with the surface of the microsphere and/or reactants bound thereto would be specific for microspheres coupled to CIAP, a control experiment was performed with BSA-coupled microspheres in the presence of ELF® 97 alcohol. The reactions conditions were 200,000 BSA-microspheres, 100 μM ELF® 97 alcohol, 1×TE, 37° C. 1 hr, 1150 rpm, light-protected, 100 μL reaction volume. Images 88 and 90 shown in FIG. 5 are representative confocal micrographs at 100×. FIG. 5 illustrates that ELF® 97 alcohol does not colocalize with the BSA-microspheres, but rather seems to form microsphere-free crystalline particles. We believe that because the ELF® 97 alcohol generated de novo by the microsphere-coupled CIAP is water-insoluble, it is thermodynamically favorable to bind to the closest hydrophobic surface, as opposed to diffusing through the aqueous reaction buffer to interact with other microspheres or surfaces. Since the ELF® 97 alcohol in this experiment was not generated at the bead surface, but rather was added to the free aqueous environment, it precipitated rather than bind to the bead surface.

### EXAMPLE 7

### Fluorescence Intensity Correlates with ELF® 97 Alcohol Crystal Matrix Complexity

During microscope imaging, it was observed that decreasing concentrations of ELF® 97 phosphate substrate used in the reaction resulted in less ELF® 97 alcohol bound at the microsphere surface and/or reactants bound thereto. Furthermore, the size and density of the ELF® 97 alcohol crystal matrix on the surface of the microsphere was proportional to the observed intensity of fluorescence. To verify this observation, we imaged 3 reactions performed with decreasing concentrations of ELF® 97 phosphate substrate (images 92, 94, and 96 shown in FIG. 6) at the same camera setting on the inverted fluorescence microscope. In particular, reactions conditions were 5,000 CIAP-microspheres, 500 μM, 250 μM, or 125 μM ELF® 97 phosphate, for images 92, 94, and 96, respectively, 1×TE, 37° C. 1 hr, 1150 rpm, light-protected, 100 μL reaction volume. Images are representative and cropped from 400× epifluorescence micrographs. All images were taken at the same gain (2.7) and exposure (1.19 ms for ELF® 97 alcohol, 32.6 ms for microspheres) camera settings. As shown in FIG. 6, images 92, 94, and 96 show that decreasing ELF® 97 phosphate concentration resulted in less ELF® 97 alcohol crystal on the bead and therefore diminished fluorescence intensity. In addition, fluorescence intensity is proportional to the ELF® 97 alcohol crystal matrix complexity. Note that in the image for the experiment containing 125 μM ELF® 97 phosphate (image 96), no ELF® 97 alcohol is observed on the microsphere. However, ELF® 97 alcohol is in fact on the microsphere but could not be detected by the camera at this exposure.

Further modifications and alternative embodiments of various aspects of the invention may be apparent to those skilled in the art in view of this description. For example, methods, products, and kits for identifying an analyte in a sample are provided. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of performing an assay to identify an analyte in a sample, comprising:
combining the sample with a set of beads, at least some of which are adapted to couple to the analyte in the sample;
mixing an enzyme with the sample and bead combination, the enzyme being adapted to couple to the analyte;
interacting a substrate with the enzyme where a property of the substrate is modified by the enzyme and the modified substrate is attracted to the beads; and
illuminating the beads to identify modified substrate attracted to the beads.

2. The method of claim 1, wherein a subset of the the beads includes a first reactant, and in the combining step (1) the analyte couples to the first reactant.

3. The method of claim 2, wherein the first reactant is an antibody and the analyte is an antigen.

4. The method of claim 1, wherein the mixing step (2) includes adding a second reactant with the sample and beads, whereby the second reactant couples to the analyte and the enzyme couples to the second reactant.

5. The method of claim 4, wherein the analyte is an antigen and the second reactant is an antibody.

6. The method of claim 1, wherein in said interacting step (3) the substrate being generally soluble and the modified substrate being less soluble.

7. The method of claim 1, wherein said illuminating step (4) is performed by a flow cytometer and said modified substrate emits fluorescence light when illuminated at a certain wavelength by said flow cytometer.

8. The method of claim 7, wherein said unmodified substrate emits at a different wavelength when illuminated.

9. The method of claim 2, wherein said first reactant is an antigen, antibody or olignucleotide.

10. The method of claim 1, wherein said beads include a dye that emits fluorescent light when illuminated.

11. A method for identifying an analyte in a sample, comprising:
combining the sample with a first reactant capable of coupling to the analyte, wherein the first reactant is coupled to beads;
mixing an additional reactant with the beads, wherein the additional reactant is capable of coupling to the analyte, and wherein an enzyme is attached to the additional reactant;
combining a substrate with the beads and reactant and enzyme mixture, wherein the substrate is capable of interacting with the enzyme to form a modified substrate wherein the solubility of the modified substrate changes causing the modified substrate to bind to a surface of the beads and/or the reactants bound to the beads; and
identifying the analyte in the sample by detecting the bound modified substrate.

12. The method of claim 11, wherein a second reactant is coupled to the analyte and the first reactant couples to the second reactant.

13. The method of claim 11, the identifying step including illuminating the mixture with a flow cytometer whereby the bound modified substrate emits fluorescence when illuminated.

14. A kit configured for use in flow cytometry for identifying an analyte in a sample, comprising:
a first reactant capable of specifically coupling to the analyte, wherein the first reactant is coupled to beads;
a second reactant capable of specifically coupling to the analyte, the second reactant including an enzyme attached to the second reactant; and
a substrate capable of specifically interacting with the enzyme to form a modified substrate, wherein if the substrate interacts with the enzyme the solubility of the substrate changes and the modified substrate will be attracted to the surface of the beads.

15. The kit of claim 14, wherein the modified substrate will be attracted to reactants bound to the beads.

16. The kit of claim 14, the modified substrate being capable of fluorescence emission when illuminated at a certain wavelength.

17. The kit of claim 14, wherein said first and second reactants are antibodies and said analyte is an antigen.

18. A product of a method for identifying an analyte in a sample by flow cytometry, comprising:
a first reactant coupled to the analyte, wherein the first reactant is further coupled to a bead;
a second reactant coupled to the analyte wherein an enzyme is attached to the second reactant; and
a modified substrate attracted to a surface of the bead and/or the reactants bound to the bead due to an interaction between an initial substrate and the enzyme that produced a change of the initial substrate.

19. The product of claim 18, wherein the change in the initial substrate to the modified substrate is a change in solubility.

20. The product of claim 18, said modified substrate being capable of fluorescence emission when illuminated at a certain wavelength during flow cytometry.
